(19) United States
(12) Patent Application Publication
(10) Pub. No.: US 2026/0345168 A1
(43) Pub. Date: Nov. 5, 2026

(54) Balloon Integrated Arterial Cannula and Method for Use

(71) Applicant: Seraphin Medical Tech. LLC, Cleveland, OH (US)

(72) Inventors: Zhuoqing Song, Kent, OH (US); Joseph Rubelowsky, Cleveland, OH (US); Matthew Villanueva, Cleveland, OH (US)

(73) Assignee: Seraphin Medical Tech. LLC, Cleveland, OH (US)

(21) Appl. No.: 19/194,862

(22) Filed: Apr. 30, 2025

(51) Int. Cl. A61B 17/12; A61B 17/00; A61M 25/10

(52) U.S. Cl. CPC A61B 17/12031; A61B 17/00234; A61B 17/12109; A61B 17/12136; A61M 25/10; A61B 2017/00238; A61B 2017/00305; A61B 2017/00557; A61M 2025/1095; A61M 2210/127

(57) ABSTRACT

The application discloses a method for facilitating organ perfusion in a deceased body. An arterial cannula is inserted into an aorta of the heart. The arterial cannula includes a compliance balloon. The arterial cannula is positioned so that a distal end of the arterial cannula is oriented towards a descending aorta of the heart; and the compliance balloon is inflated to completely occlude blood flow through the arch vessels of the heart, preventing blood flow through the arch vessels to the brain.

### TECHNICAL FIELD

This invention relates to the field of medical devices, specifically balloon cannulas for organ procurement.

### BACKGROUND

Organ procurement from circulatory death donors, or donation after circulatory death (DCD), is often a process that many transplant surgeons may avoid due to the inability to check the functionality of the organs before direct procurement. Normothermic Regional Perfusion (NRP) is a procedure that allows temporary perfusion of the heart and other organs in the donor's body by establishing a cardiopulmonary bypass, allowing surgeons to gather information on the function and condition of the organs. Thoracoabdominal normothermic reperfusion procedure (TA-NRP) is the process in which the thoracic and abdominal organs are perfused.

Resuscitation of the brain after the declaration of circulatory death poses ethical issues, thus a major concern in NRP is preventing restored blood flow in the heart from making its way to the brain. To prevent this, the aortic arch vessels are clamped before initiating perfusion. Immediately after the arch vessels are clamped, an aortic cannula is inserted into the aorta in order to re-introduce circulation.

The process of clamping the aortic arch vessels takes place during a crucial time in the procurement process; the heart is revived before the heart and other organs experience noticeable damages from lack of blood flow and adequate temperature. The clamping of the arch vessels takes valuable time, and the actual clamping of the arch vessels is performed with limited visibility by the transplant surgeons, adding additional challenges and complexity.

### BRIEF DESCRIPTION

According to one aspect, a method for facilitating organ perfusion in a deceased body includes inserting an arterial cannula into an aorta of the heart. The arterial cannula includes a compliance balloon. The arterial cannula is positioned so that the distal end of the arterial cannula is oriented towards the descending aorta of the heart. The compliance balloon is inflated to completely occlude blood flow through the arch vessels of the heart, preventing blood flow through the arch vessels to a brain.

According to another aspect, a method for normothermic regional perfusion includes inserting an arterial cannula into the ascending aorta of the heart, where the arterial cannula includes a compliance balloon around its distal end. The arterial cannula is positioned so that the compliance balloon is oriented towards the aortic arch of the heart. The compliance balloon is inflated until blood flow to the arch vessels is completely occluded, preventing blood flow to the brain through the arch vessels. The blood flow to the arch vessels is permanently blocked, and the compliance balloon is deflated to reperfuse the heart.

According to a further aspect, an arterial balloon cannula includes an arterial cannula for insertion into an aortic arch of a heart, a compliance balloon around a distal end of the arterial cannula for occluding arch vessels, and a fluid outlet at the distal end of the arterial cannula beyond the compliance balloon. The compliance balloon is inflated within the aortic arch, completely occluding blood flow through arch vessels of the heart to a brain. Upon externally sealing the arch vessels, the compliance balloon is deflated.

### BRIEF DESCRIPTION OF THE DRAWINGS

The accompanying drawings, which are incorporated in and constitute a part of the specification, illustrate various systems, methods, devices, and other embodiments of the disclosure. It will be appreciated that the illustrated element boundaries (e.g., boxes, groups of boxes, directional lines, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIG. 1A illustrates an arterial cannula with a compliance balloon in the aortic arch of the heart where the compliance balloon is deflated according to an exemplary embodiment;

FIG. 1B illustrates the arterial cannula and the compliance balloon in the aortic arch of the heart of FIG. 1A where the compliance balloon is inflated according to an exemplary embodiment;

FIG. 2 is schematic diagram illustrating a heart-lung machine as used in TA-NRP according to an exemplary embodiment;

FIG. 3 is a flow diagram of a method of using the balloon integrated arterial cannula according to an exemplary embodiment;

FIG. 4 is a flow diagram of a method of using the balloon integrated arterial cannula according to another exemplary embodiment;

FIG. 5A illustrates the arterial cannula with the compliance balloon inflated as shown in FIG. 1B, but placed in the descending aorta according to an alternate exemplary embodiment;

FIG. 5B illustrates the arterial cannula with the compliance balloon inflated as shown in FIG. 1B, but placed in the descending aorta and including an alternative cannulation point according to an alternate exemplary embodiment;

FIG. 6A is side cross section of the structure of an arterial balloon cannula according to an exemplary embodiment;

FIG. 6B is an alternative view of the arterial balloon cannula of 6A;

FIG. 6C is a top view of the arterial balloon cannula of FIG. 6A; and

FIG. 6D is a transverse cross section of the arterial balloon cannula of FIG. 6A.

### DETAILED DESCRIPTION

The balloon integrated arterial cannula and method for use facilitates organ perfusion in a deceased body and includes inserting an arterial cannula into an aorta of a heart. The arterial cannula includes a compliance balloon. Once inserted into the ascending aorta, the arterial cannula is positioned so that the distal end of the arterial cannula is oriented towards the descending aorta of the heart, and the arterial cannula is inflated so the compliance balloon completely occludes blood flow through aortic arch vessels, thereby preventing blood flow through the arch vessels to the deceased body's brain. The balloon integrated arterial cannula and method of use provides the benefit of rapid arch vessel occlusion that removes the need to immediately clamp the aortic arch vessels prior to initiating perfusion. Additionally, the decompression of the heart that results from the occlusion provides additional space and ability to maneuver for clamping or otherwise sealing the arch vessels.

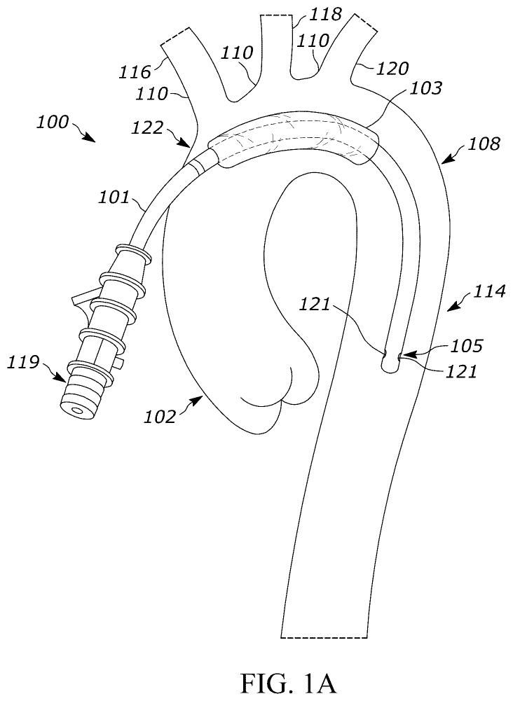

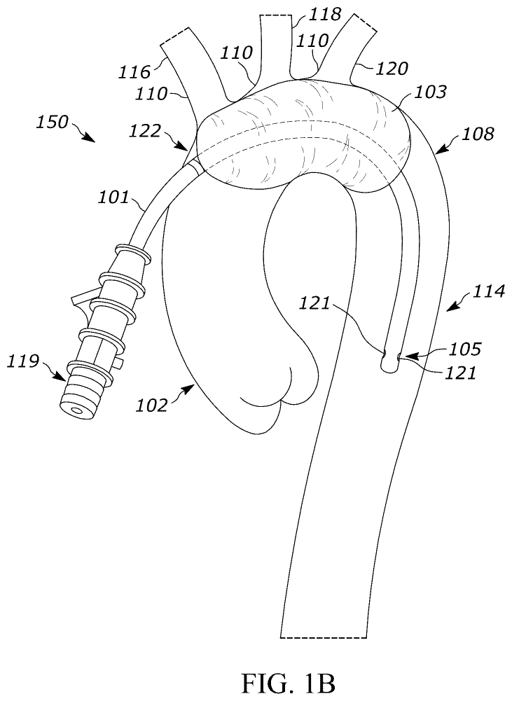

Referring now to the drawings, wherein the showings are for purposes of illustrating one or more exemplary embodiments and not for purposes of limiting the same, FIG. 1A illustrates a view 100 of the aorta with the balloon integrated arterial cannula 101 with compliance balloon 103 deflated therein. The view 100 of the aorta includes the ascending aorta 102, the aortic arch 108, and the descending aorta 114. The aortic arch 108, as used herein, is defined as the space between the ascending aorta 102 and descending aorta 114, where the arch vessels 110 of the heart are located. The arch vessels 110 may include the brachiocephalic artery 116 (also known as the innominate artery) the left common carotid artery 118, and the left subclavian artery 120. The arch vessels 110 are also referred to as head vessels. The arch vessels 110 may include three separate arch vessels, or may include at least one arch vessel, or may be a different anatomical configuration.

The arterial cannula 101 may be a cannula or a catheter with one or more channels. The arterial cannula 101 may be configured to have multiple channels, including channels for air, liquids, guidewires, or other purposes. The arterial cannula 101 may be of a size and flexibility capable of navigating the aortic arch.

The arterial cannula 101 includes a compliance balloon 103. The compliance balloon may be wrapped around the arterial cannula 101. The compliance balloon may be located around the distal end 105 of the arterial cannula 101 or may be located between the proximate end 119 and the distal end 105 of the arterial cannula 101. The compliance balloon 103 may be silicone, polyurethane, latex, or another flexible material. The compliance balloon 103 may be inflated to a diameter of up to 45 mm. The proximate end 119 of the arterial cannula 101 may also include a control mechanism for the arterial cannula 101, a blood inlet, a de-air port, among other components. The compliance balloon 103 may be tightly wrapped around the arterial cannula 101 or otherwise attached prior to an initial inflation.

The arterial cannula 101 may be inserted into a cannulation point 122 directly or indirectly into the ascending aorta 102. The cannulation point 122 for the arterial cannula 101 may also be located in the aortic arch 108, or at a point between the ascending aorta 102 and the left subclavian artery 120. An introducer, dilator, or guidewire may be used to aid in arterial cannula insertion (not shown). The distal end 105 of the arterial cannula 101 may be oriented toward the descending aorta 114. The arterial cannula 101 may be positioned so that the distal end 105 of the cannula is directed into the aortic arch 108 or further into the descending aorta 114. The arterial cannula 101 may allow fluid flow through one or more fluid outlets 121 to allow for perfusion of the abdominal organs.

FIG. 1B illustrates a view 150 of the aorta with the balloon integrated arterial cannula 101 with compliance balloon 103 inflated therein. The compliance balloon 103 is inflated within the aortic arch 108 to completely occlude blood flow through the arch vessels 110. Three arch vessels 110 may be occluded by the compliance balloon 103. As used herein, “complete occlusion” is sufficient occlusion to prevent brain flow to the brain. Complete occlusion also may mean sufficient occlusion to prevent brain resuscitation, which includes 100% prevention of blood flow through the arch vessels and also includes occlusion of over 90% of blood flow. Blood or blood flow includes oxygenated blood and may include the addition of preservation additives known in the art. Blood or blood flow may also include deoxygenated blood and may include the addition of preservation additives.

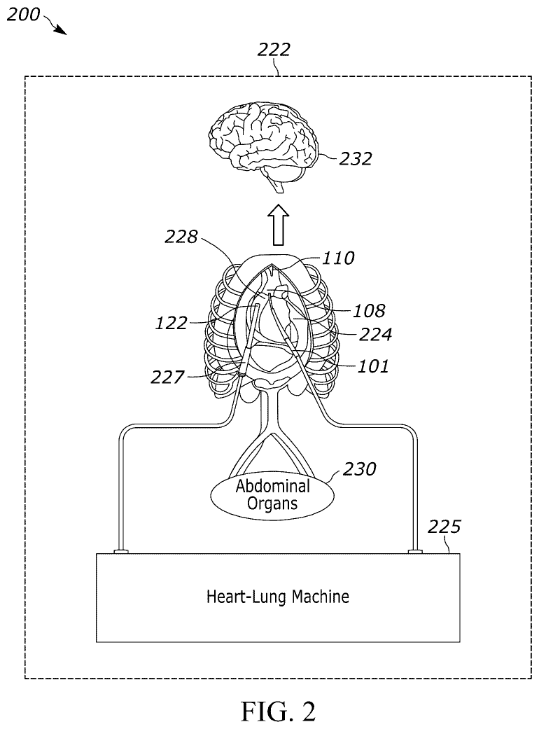

When the compliance balloon 103 is inflated, and complete occlusion is accomplished, the heart may not beat and may be decompressed when used in combination with a venous cannula inserted into the right atrium of the heart (shown and described below in FIG. 2). When the heart is decompressed and the abdominal organs are perfused via the arterial cannula 101, the arch vessels 110 may be externally sealed. Externally sealing may be performed via clamps, ties, sutures, staples, dissection, or other methods.

When the arch vessels 110 are externally sealed, the compliance balloon 103 may be deflated to allow for reperfusion of the heart. When the compliance balloon 103 is deflated, the heart may resume beating. The compliance balloon 103 may also be deflated for removal of the arterial cannula 101. The compliance balloon 103 may be inflated or deflated as necessary.

The arterial balloon cannula and method disclosed herein may be used in thoracoabdominal normothermic regional perfusion (TA-NRP) procedures. Accordingly, reference is now made to FIG. 2, which illustrates a heart-lung machine as used in TA-NRP according to an exemplary embodiment 200. The method for TA-NRP may also include opening the chest cavity of a deceased donor body 222, to access the heart 224, where the donor body 222 was derived from a circulatory death. For TA-NRP, a heart-lung machine 225 may be used. In an exemplary embodiment, a heart-lung machine 225, also known as a cardiopulmonary bypass pump, provides for extracorporeal oxygenation and circulation. After opening the chest cavity, a venous cannula 227 may be inserted into the right atrium 228 of the heart. The venous cannula 227 drains deoxygenated blood and decompresses the heart 224. The arterial cannula 101 with attached compliance balloon 103 provides oxygenated blood to perfuse or reperfuse the abdominal organs 230, while occluding blood flow to the brain 232. The oxygenated blood flow to arch vessels 110 may be completely occluded while the abdominal organs 230 are simultaneously perfused or reperfused. With the compliance balloon 103 on the arterial cannula 101 inflated, the heart 224 stops beating and the venous cannula 227 decompresses the heart.

After the heart has been decompressed and the abdominal organs perfused, the arch vessels 110 may be externally sealed. Externally sealing may be performed via clamps, ties, sutures, staples, dissection, or other methods. The arch vessels 110 may also be permanently detached from the brain via dissection for organ transplant or other purposes. While the heart is decompressed, parts of the aortic arch 108, the ascending aorta 102, and the arch vessels 110 are softer and easier to access or manipulate, allowing for easier access for clamping or otherwise externally sealing the arch vessels 110.

Deflating the compliance balloon 103 allows oxygenated blood flow to reperfuse the heart, allowing the heart to begin beating. This preserves the heart for future assessment or donation. Assessment of the heart 224 may include visual inspection or other diagnostics for viability. While perfused, other organs may also be assessed for donation viability. Perfusion blood flow may also be paused for visual inspection of the heart, which may include also the use of a transesophageal echocardiogram (TEE), and/or handheld echo probes.

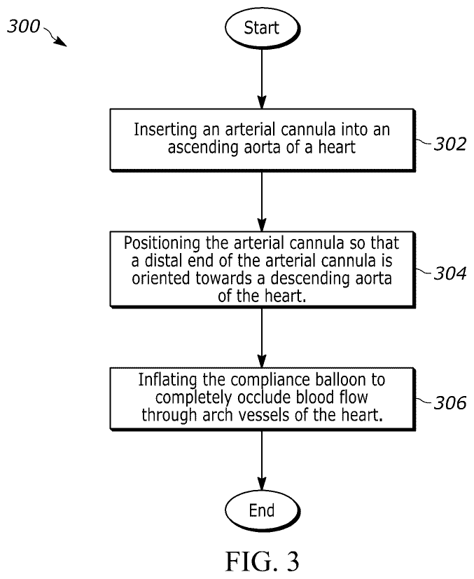

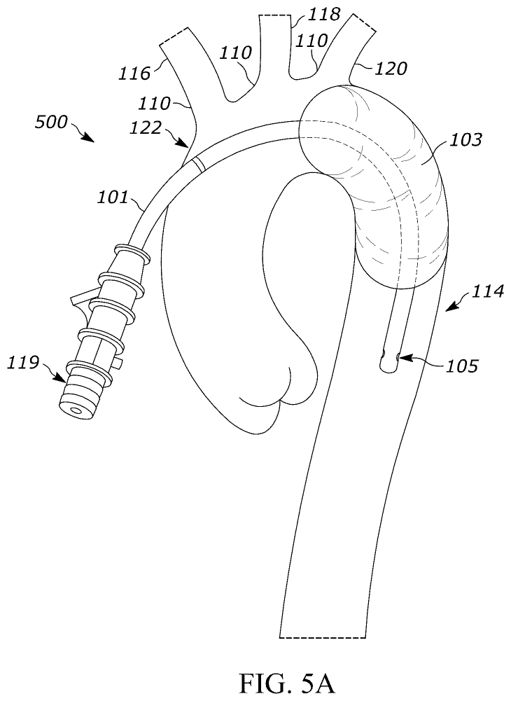

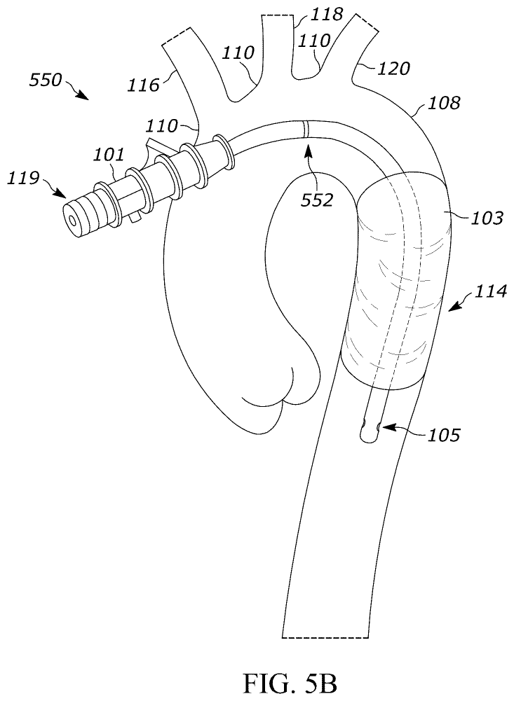

Referring now to FIG. 3, a method 300 for using the balloon integrated arterial cannula will now be described with further reference to the components and methods of FIGS. 1 and 2. At block 302, the method 300 includes inserting an arterial cannula into an ascending aorta of a heart. As discussed above, the arterial cannula 101 includes a compliance balloon 103. At block 304, the method 300 includes positioning the arterial cannula 101 so that a distal end of the arterial cannula is oriented towards a descending aorta of the heart. In some embodiments, the arterial cannula 101 may be positioned so that the distal end 105 of the cannula is located in the descending aorta 114 (see FIG. 5). In some embodiments, the arterial cannula 101 may be positioned so that the distal end 105 of the cannula is located in the aortic arch. At block 306, the method 300 includes inflating the compliance balloon to completely occlude blood flow through arch vessels of the heart, thereby preventing blood flow through the arch vessels to a brain. As described above, preventing blood flow to the brain may be complete occlusion.

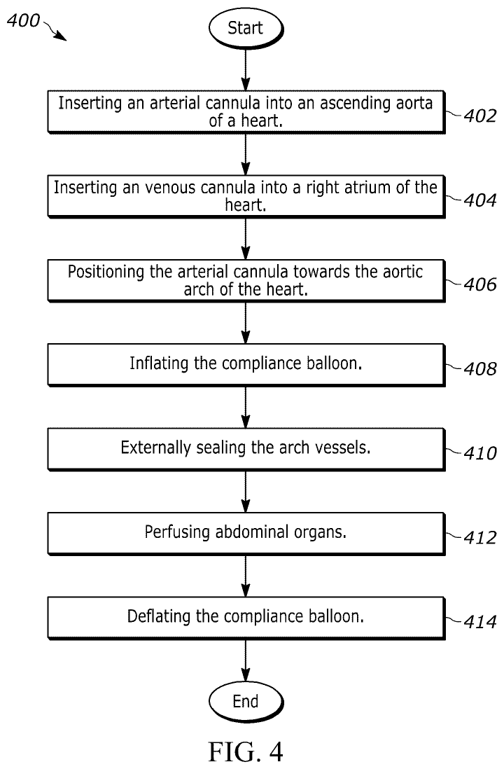

Referring now to FIG. 4, another method 400 of using the balloon integrated arterial cannula will now be described with further reference to the components of FIGS. 1 and 2. The method 400 at block 402 includes inserting an arterial cannula into the ascending aorta of the heart. In some embodiments, the arterial cannula 101 may be positioned so that the distal end 105 of the cannula is directed further into the descending aorta 114 (see FIG. 5). At block 404, the method 400 includes inserting a venous cannula into the right atrium of the heart. For example, the venous cannula 227 is inserted into the right atrium 228 of the heart. The venous cannula 227 drains deoxygenated blood.

At block 406, the method 400 includes positioning the arterial cannula towards the aortic arch of the heart. At block 408, the method 400 includes inflating the compliance balloon. For example, with the compliance balloon 103 of the arterial cannula 101 inflated, the venous cannula 227 decompresses the heart. At block 410, the method 400 includes externally sealing the arch vessels. As discussed above, externally sealing may be performed via clamps, ties, sutures, staples, dissection, or other methods. At block 412, the method 400 includes perfusing the abdominal organs. At block 414, the method 400 includes deflating the compliance balloon. For example, after deflating the compliance balloon 103, the oxygenated blood flow may reperfuse the heart. Thus, oxygenated blood flow to arch vessels 110 may be completely occluded while the abdominal organs 230 are simultaneously perfused or reperfused

Referring now to FIG. 5A, a view 500 of another alternative placement of the arterial cannula 101 will be described with further reference to the components of FIGS. 1 and 2. In this alternate embodiment, the compliance balloon 103 is inflated within the descending aorta 114. In this embodiment, the compliance balloon may be in the descending aorta 114, including any area between the aortic arch and the abdominal aorta (not shown). The inflated compliance balloon 103 in the descending aorta 114 may completely occlude blood flow to the arch vessels 110, the brachiocephalic artery 116, the left common carotid artery 118, and the left subclavian artery 120, and the brain 232. In this embodiment, the arterial cannula 101 is inserted at cannulation point 122 in the ascending aorta 102 as described in FIGS. 1 and 2.

Referring now to FIG. 5B, a view 550 of an alternative placement of the arterial cannula 101 will be described with further reference to the components of FIGS. 1 and 2. In this alternate embodiment, the arterial cannula 101 may be inserted into an alternate cannulation point 552 in the aortic arch 108. The cannulation point 552 for the arterial cannula 101 may be between the left common carotid artery 118 and the left subclavian artery 120. In this embodiment, the compliance balloon 103 may be in the descending aorta, including any area between the aortic arch 108 and the abdominal aorta (not shown). The inflated compliance balloon 103 in the descending aorta 114 may completely occlude blood flow to the arch vessels 110, the brachiocephalic artery 116, the left common carotid artery 118, and the left subclavian artery 120, and the brain 232.

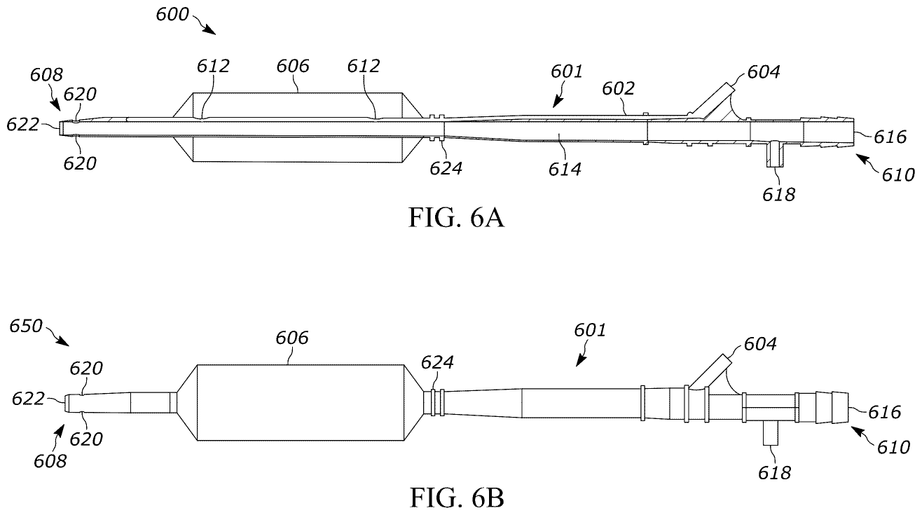

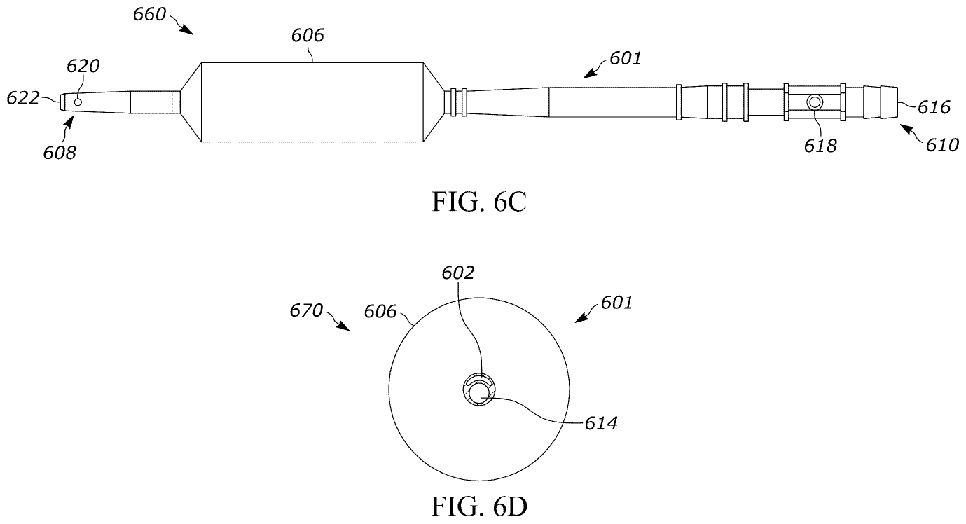

FIG. 6A, shows a side cross section view 600 of the structure of the arterial cannula 601. The arterial cannula 601 may be inserted into an aortic arch of a heart. The arterial cannula 601 may be a cannula, catheter, capillary or other type of tube with one or more channels. The arterial cannula 601 may be configured to have multiple channels, including channels for air, liquids, introducers, dilators, or guidewires among others. Such channels may be in fluidic connection or may be separate. The arterial cannula 601 may be of a size or flexibility capable of navigating the aortic arch. The arterial cannula 601 may have a maximum diameter of 7.7 mm or may have a maximum diameter of another size based upon the parameters of the donor anatomy. The arterial cannula 601 may include fluid or air flow through one or more channels. The arterial cannula 601 includes an air channel 602 and an air inlet 604 to allow the inflation or deflation of the compliance balloon 606.

A compliance balloon 606 is wrapped around a distal end 608 of the arterial cannula 601 that may occlude the arch vessels. The compliance balloon 606 may be located directly at the distal end 608 of the cannula, or the compliance balloon 606 may be located at any point between the distal end 608 of the arterial cannula 101 and the proximate end 610 of the arterial cannula 601. The compliance balloon 606 may be tightly wrapped around an arterial cannula 601 prior to inflation. The compliance balloon 606 may be inflated, as shown in FIG. 6A. The compliance balloon 606 may be silicone, polyurethane, latex, or another flexible material. The compliance balloon 606 may range from 20 mm to 45 mm in diameter. The compliance balloon 606 may be inflated via a syringe, a pump, or another inflation method. Uses of the compliance balloon 606 includes occluding the arch vessels.

After insertion of the arterial cannula 601 into the aorta, a syringe, pump, or other inflation method may be attached to the air inlet 604 of the arterial cannula 601 and used to inflate the compliance balloon 606. The air inlet 604 may extend outward from the arterial cannula 601 at an acute angle, adjacent to the proximate end 610 of the cannula, or the air inlet 604 may extend outward at another angle.

In this embodiment, the air channel 602 has an air inlet 604 and inflation ports 612, with the compliance balloon 606 covering multiple inflation ports 612 outside of the arterial cannula 601. The inflation ports 612 may include one or more holes to allow air out of the air channel 602 to inflate or deflate the compliance balloon 606. The compliance balloon 606 may be inflated within the aortic arch, thereby completely occluding blood flow through the arch vessels of the heart to the brain. Inflation of the compliance balloon 606 may occur until the compliance balloon 606 cannot further inflate or until the arch vessels are fully occluded. Upon externally sealing the arch vessels, the compliance balloon 606 may be deflated. Deflation of the compliance balloon 606 may allow for reperfusion of the heart.

The arterial cannula 601 also may include a fluid channel 614, where the fluid channel 614 has a fluid inlet 616, located at the proximate end 610 of the arterial cannula. The fluid inlet 616 may be the opening of the proximate end 610 of the cannula. The fluid inlet 616 may allow for connection to a heart-lung machine. When in use, blood flow through the fluid channel 614 may reperfuse abdominal organs. The arterial cannula 601 also may include a de-air port 618, located at the proximate end 610 of the arterial cannula 601, which may allow for the removal of air bubbles from the fluid channel 614. The de-air port 618 may extend perpendicularly outward from the arterial cannula 601, adjacent to the proximate end 610 of the cannula, or the de-air port 618 may extend outward at an acute angle. The arterial cannula 601 also includes at least one fluid outlet 620 located at the distal end 608 of the arterial cannula beyond the compliance balloon 606. The fluid outlet 620 is in fluidic connection with the fluid channel 614. The fluid outlet 620 may be an opening at the cannula tip 622 of the arterial cannula. Or as shown in the embodiment of FIG. 6A., the fluid outlet 620 may include one or more holes on the sides of the cannula between the cannula tip 622 and the compliance balloon 606. The arterial cannula 601 may include an optional movable stopper 624, located adjacent to the compliance balloon 606, between the compliance balloon 606 and the proximate end 610 of the arterial cannula.

FIG. 6B shows an alternative view 650 of the structure of the arterial cannula 601, showing the arterial cannula in full. The compliance balloon 606, the fluid inlet 616, de-air port 618, air inlet 604, fluid outlets 620, and optional movable stopper 624 are shown.

FIG. 6C shows a top view 660 of the structure of the arterial cannula 601. The compliance balloon 606, air inlet 604, optional movable stopper 624, and fluid outlets 620 are shown.

FIG. 6D shows a transverse cross section view 670 of the arterial cannula 601. FIG. 6D shows, from outside to inside, the compliance balloon 606, followed by the air channel 602, and fluid channel 614.

A person of ordinary skill in the art would understand the arterial cannula 601 could include other components not shown in FIGS. 6A-6D. For example, a removable introducer may be threaded through the fluid channel 614. The introducer may also be threaded through other channels. The introducer could be used to facilitate entry of the arterial cannula 601 to the aorta. The introducer may also be a dilator. A dilator could also be used to stretch an opening to allow insertion of the arterial cannula 601 into the ascending aorta, or to facilitate navigation of the cannula within the aortic arch. The introducer reversibly connects to the proximate end 610 of the arterial cannula 601 via a soft connector. The soft connector may facilitate a connection between the introducer and the arterial cannula 601. Finally, a guidewire may be threaded through the introducer, or through other channels. The guidewire may be used to facilitate navigation of the cannula within the aortic arch.

It will be appreciated that various embodiments of the above-disclosed and other features and functions, or alternatives or varieties thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A method for facilitating organ perfusion in a deceased body, comprising:
inserting an arterial cannula into an aorta of a heart, wherein with the arterial cannula includes a compliance balloon;
positioning the arterial cannula so that a distal end of the arterial cannula is oriented towards a descending aorta of the heart; and
inflating the compliance balloon to completely occlude blood flow through arch vessels of the heart, thereby preventing blood flow through the arch vessels to a brain.

2. The method of claim 1, wherein the arterial cannula is inserted into an ascending aorta.

3. The method of claim 1, wherein the compliance balloon is inflated within an aortic arch.

4. The method of claim 1, wherein the arterial cannula is inserted into an aortic arch.

5. The method of claim 4, wherein the compliance balloon is inflated in the descending aorta.

6. The method of claim 1, wherein the compliance balloon completely occludes a brachiocephalic artery, a left common carotid artery, and a left subclavian artery.

7. The method of claim 1, wherein the compliance balloon is around the distal end of the arterial cannula.

8. The method of claim 1, further comprising inserting a venous cannula into a right atrium of the heart, wherein the venous cannula decompresses the heart.

9. The method of claim 1, further comprising deflating the compliance balloon to reperfuse the heart.

10. The method of claim 1, wherein the method is used in normothermic regional perfusion.

11. The method of claim 1, further comprising perfusing abdominal organs.

12. The method of claim 11, wherein blood flow to the arch vessels is completely occluded while simultaneously perfusing the abdominal organs.

13. The method of claim 1, wherein three arch vessels are occluded by the compliance balloon.

14. A method for organ perfusion, comprising:
inserting an arterial cannula into an ascending aorta of a heart, wherein the arterial cannula includes a compliance balloon around a distal end of the arterial cannula;
positioning the arterial cannula so that the compliance balloon is oriented towards an aortic arch of the heart;
inflating the compliance balloon until blood flow to arch vessels is completely occluded, wherein the blood flow through the arch vessels to a brain is prevented;
permanently blocking the blood flow through the arch vessels; and
deflating the compliance balloon to reperfuse the heart.

15. The method of claim 14, further comprising inserting a venous cannula into a right atrium of the heart.

16. The method of claim 14, further comprising perfusing abdominal organs.

17. The method of claim 14, wherein the arch vessels are externally sealed.

18. An arterial balloon cannula, comprising:
an arterial cannula for insertion into an aortic arch of a heart;
a compliance balloon around a distal end of the arterial cannula for occluding arch vessels; and
at least one fluid outlet at the distal end of the arterial cannula beyond the compliance balloon, wherein the compliance balloon is inflated within the aortic arch, thereby completely occluding blood flow through the arch vessels of the heart to a brain, and, upon externally sealing the arch vessels, the compliance balloon is deflated.

19. The arterial balloon cannula of claim 18, wherein the arterial cannula includes an air channel for inflation and deflation of the compliance balloon, wherein deflating the compliance balloon reperfuses the heart.

20. The arterial balloon cannula of claim 18, wherein the arterial cannula includes a fluid channel, wherein blood flow through the fluid channel reperfuses abdominal organs.
